(19) United States
(12) Patent Application Publication
(10) Pub. No.: US 2025/0111948 A1
(43) Pub. Date: Apr. 3, 2025

(54) POST OPERATIVE INFECTION AND PRESSURE INJURY PREDICTOR

(71) Applicant: Hill-Rom Services, Inc., Batesville, IN (US)

(72) Inventors: Autumn S. Thompson, Philadelphia, PA (US); Kathryn Piston, Syracuse, NY (US)

(21) Appl. No.: 18/885,032

(22) Filed: Sep. 13, 2024

Related U.S. Application Data
(60) Provisional application No. 63/586,023, filed on Sep. 28, 2023.

(51) Int. Cl. G16H 50/30; G16H 10/60; G16H 40/20

(52) U.S. Cl. CPC G16H 50/30; G16H 10/60; G16H 40/20

(57) ABSTRACT

A system for improving a prediction of post-operative patient risks is disclosed. The system receives data, and calculates at least one post-operative score based on the data. The at least one post-operative score is at least partially calculated by monitoring interactions between a patient and other persons. The system issues an alert when the at least one post-operative score exceeds a threshold value. The system generates at least one recommendation for improving the at least one post-operative score. The system presents a control on a graphical user interface for viewing the at least one post-operative score or the at least one recommendation.

### BACKGROUND

Patients undergo surgical procedures within clinical care environments to treat various medical conditions. Patients that undergo surgical procedures are at risk for numerous adverse medical events. Two examples of adverse medical events that can occur during or after a surgical procedure are pressure injuries (PIs) or healthcare-associated infections (HAIs). PIs and HAIs can severely impact a patient's health, increase the risk of negative healthcare outcomes including even death, and greatly extend the patient's stay in the clinical care environment.

### SUMMARY

In general terms, the present disclosure relates systems and methods for improving a prediction of post-operative patient risks within a clinical care setting. In one possible configuration, a monitoring system receives input data from one or more systems, analyzes the input data, generates system outputs, and issues an alert and/or recommendation when one or more post-operative scores exceed a threshold value. Various aspects are described in this disclosure, which include, but are not limited to, the following aspects.

One aspect relates to a system for improving a prediction of post-operative patient risks, the system comprising: at least one processing device; and a memory device storing instructions which, when executed by the at least one processing device, cause the at least one processing device to: receive data; calculate at least one post-operative score based on the data, wherein the at least one post-operative score is at least partially calculated by monitoring interactions between a patient and other persons; issue an alert when the at least one post-operative score exceeds a threshold value; generate at least one recommendation for improving the at least one post-operative score; and present a control on a graphical user interface for viewing the at least one post-operative score or the at least one recommendation.

Another aspect relates to a method for improving a prediction of post-operative infection and pressure injuries, the method comprising: receiving data from at least one monitoring device; calculating a post-operative score based on the data received from the at least one monitoring device, wherein the post-operative score is at least partially calculated by monitoring interactions between a patient and other persons; issuing an alert when the post-operative score exceeds a threshold value; generating at least one recommendation for improving the post-operative score; and presenting a control on a graphical user interface for viewing at least one of the post-operative score and the at least one recommendation.

Another aspect relates to a non-transitory computer readable storage medium storing instructions, which when executed by a computing device, causes the computing device to: receive an alert including at least one post-operative score; receive at least one recommendation for improving the at least one post-operative score; display the at least one post-operative score and the at least one recommendation; request an input from the caregiver via a user interface to approve the at least one recommendation; and implement the recommendation based on the input from the caregiver.

### DESCRIPTION OF THE FIGURES

The following drawing figures, which form a part of this application, are illustrative of the described technology and are not meant to limit the scope of the disclosure in any manner.

FIG. 1 schematically illustrates an example of a clinical care environment that includes a monitoring system configured to calculate post-operative scores relating to a prediction of post-operative patient risks within the clinical care environment.

FIG. 2 schematically illustrates an example of the monitoring system in the clinical care environment of FIG. 1.

FIG. 3 schematically illustrates an example of a method of improving post-operative scores calculated by the monitoring system of FIG. 2.

FIG. 4 schematically illustrates examples of input data that can be generated by one or more devices and systems in the clinical care environment of FIG. 1.

FIG. 5 schematically illustrates examples of system outputs that can be generated by the monitoring system of FIG. 2.

FIG. 6 illustrates an example of an operating room equipped with various clinical care environment systems to collect input data for analysis by the monitoring system of FIG. 2.

FIG. 7 illustrates an example of a recovery room equipped with various clinical care environment systems to collect input data for analysis by the monitoring system of FIG. 2.

FIG. 8 illustrates an example of a communications device displaying an alert that includes a post-operative score generated by the monitoring system of FIG. 2.

FIG. 9 illustrates an example of a communications device displaying a recommendation corresponding to the alert in FIG. 8 to improve the post-operative score.

FIG. 10 illustrates an example of automatically implementing at least one recommendation to improve the post-operative score in the alert of FIG. 8.

### DETAILED DESCRIPTION

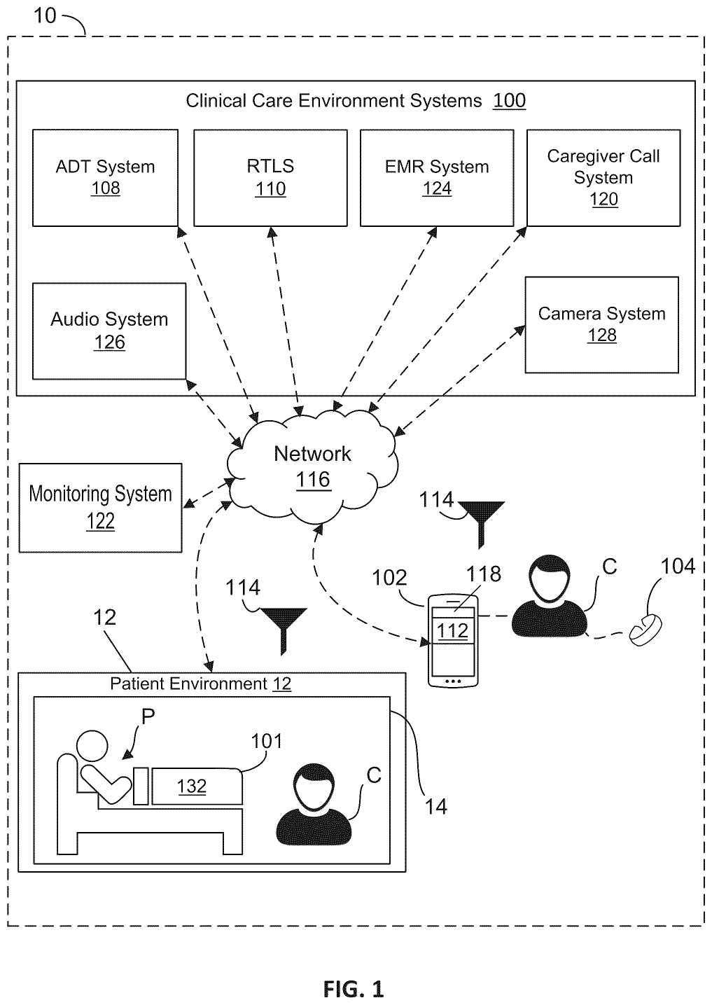

FIG. 1 schematically illustrates an example of a clinical care environment 10 that includes a monitoring system 122 configured to calculate post-operative scores relating to a prediction of post-operative patient risks within the clinical care environment 10. As will be described in further detail below, the monitoring system 122 receives input data from one or more clinical care environment systems 100 and calculates one or more post-operative scores based on an analysis of the input data.

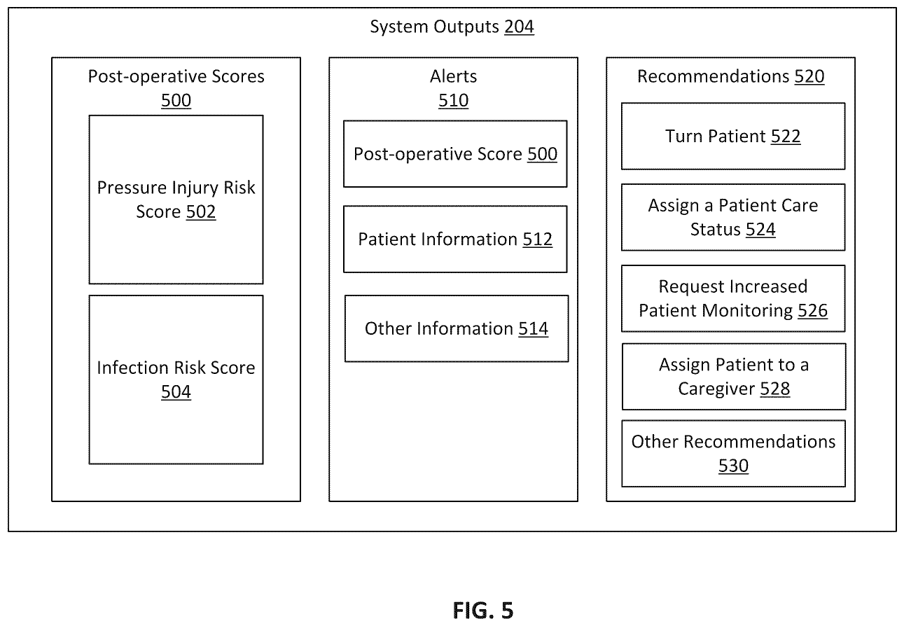

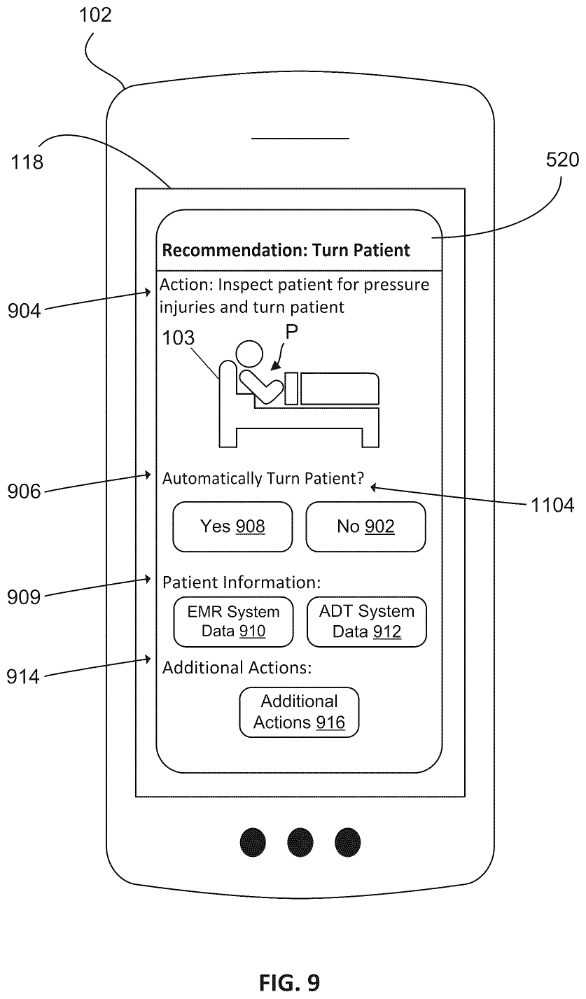

In some examples, the monitoring system 122 calculates one or more post-operative scores, compares the post-operative scores to a threshold value, generates one or more outputs (e.g., an alert or recommendation), and presents the one or more outputs to a caregiver to allow the caregiver to view the one or more outputs. The outputs can be presented to the caregiver C via a control on a graphical user interface that allows a caregiver C to view the post-operative score(s) and implement the recommendation (which is illustrated and described in further detail with respect to FIGS. 5 and 9). The monitoring system 122 is configured to receive data from clinical care environment systems 100 such as an admission, discharge, transfer (ADT) system 108, a real-time locating system (RTLS) 110, an electronical medical record (EMR) system 124, a caregiver call system 120, an audio system 126, and a camera system 128, each of which will be described in great detail below.

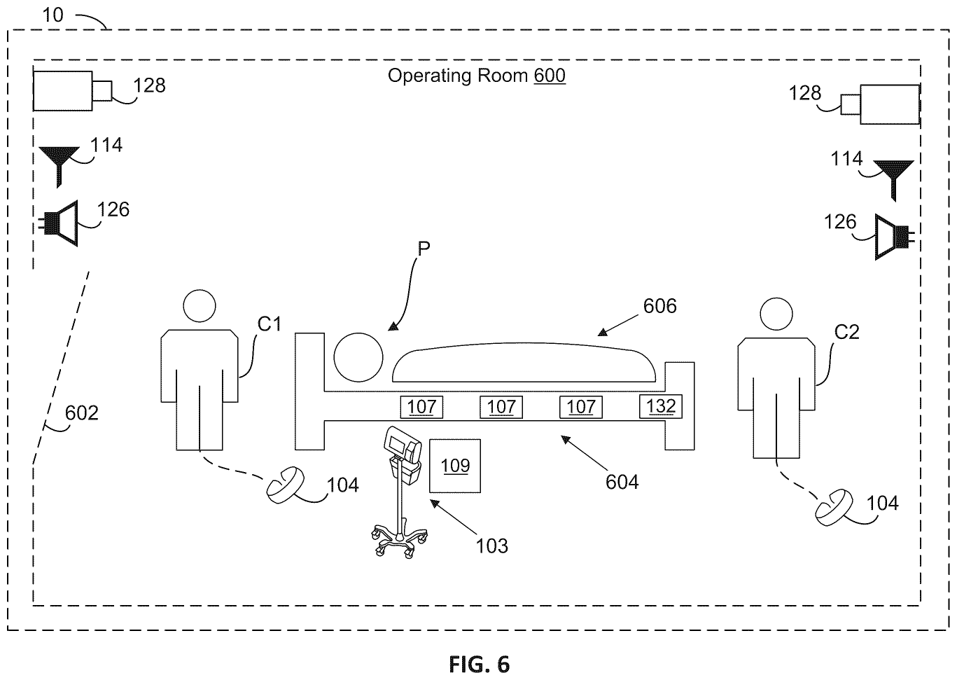

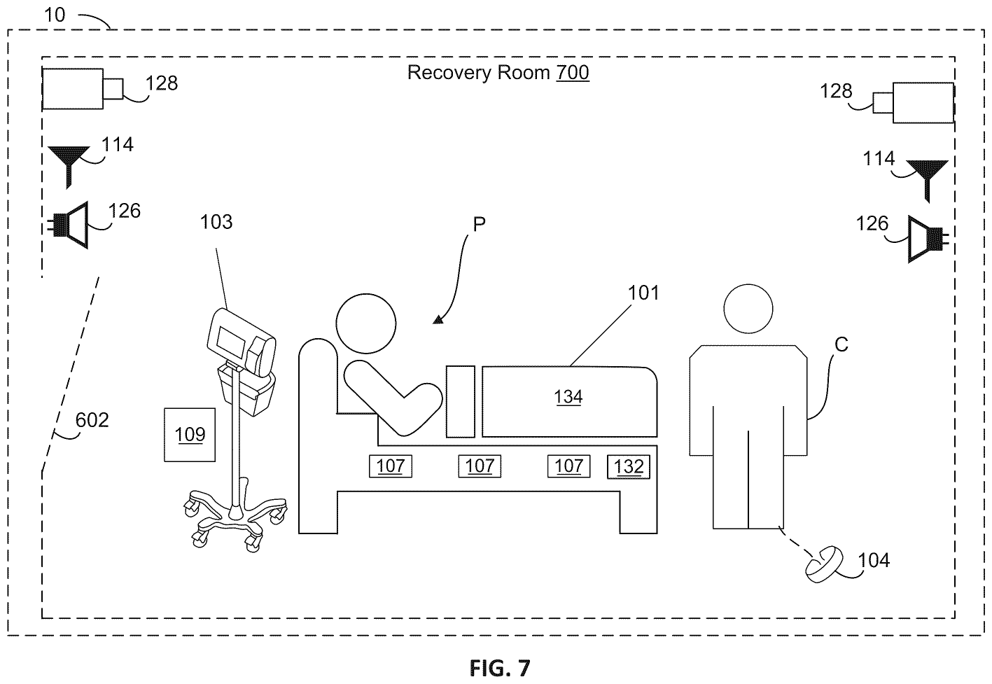

In certain examples, the clinical care environment 10 is a healthcare facility such as a hospital, a nursing home, a rehabilitation center, a long-term care facility, and the like. In certain examples, and as shown in FIG. 1, individuals within the clinical care environment 10 include one or more patients P and one or more caregivers C. As shown in FIG. 1, the patient P is located in a patient environment 12, which can include a room or other designated area within the clinical care environment 10. In one example, the patient environment 12 can include a patient room, a department (e.g., emergency department), clinic, ward, or other area within the clinical care environment 10. In another example, the patient environment 12 can include an operating room or a recovery room for housing the patient P after surgery. Furthermore, the patient environment 12 may include one or more medical devices 14 that are connected to the communications network 116. The one or more medical devices 14 may include at least one of a patient bed 101, a spot monitor 103 (as shown in FIGS. 6-7), a contact-free continuous monitoring system 132, or an infusion pump 109 (as shown in FIGS. 6-7).

A caregiver C operates a communications device 102 on which the caregiver C may receive alerts and/or recommendations from the monitoring system 122. Examples of the communications devices 102 can include smartphones, tablet computers, or other types of portable computing devices. In further examples, the communications devices 102 can include workstation computers. In some examples, a communication application 112 can be downloaded on the communications devices 102 to facilitate communication between the caregiver C and the monitoring system 122. In further examples, the communication application 112 can be a web-based or cloud-based application that is accessible on the communications devices 102.

In certain examples, the monitoring system 122 can provide communication channels allowing the caregivers C, administrators, or patients P to leave video, audio, or text messages to communicate during an emergency or discuss a patient's condition. This may include emergencies relating to infections or pressure injuries that develop after surgery. As discussed below with reference to FIGS. 5, 8, and 9, the post-operative scores can influence alerts, recommendations, and any follow-up actions required to monitor patients P. In some examples, the monitoring system 122 is an extension of the Voalte® platform available from Hillrom®.

Data that is collected from one or more clinical care environment systems 100 (described in further detail below) within the clinical care environment 10 can be transmitted to the monitoring system 122 across a communications network 116. As shown in FIG. 1, the communications devices 102 used by the caregivers C are all connected to the communications network 116. The communications network 116 can include any type of wired or wireless connections or any combinations thereof. Examples of wireless connections include Wi-Fi, Bluetooth, and broadband cellular networks including 4G or 5G. In some examples, the communications network 116 includes a broadband cellular network. In some examples the communications network 116 includes the Internet.

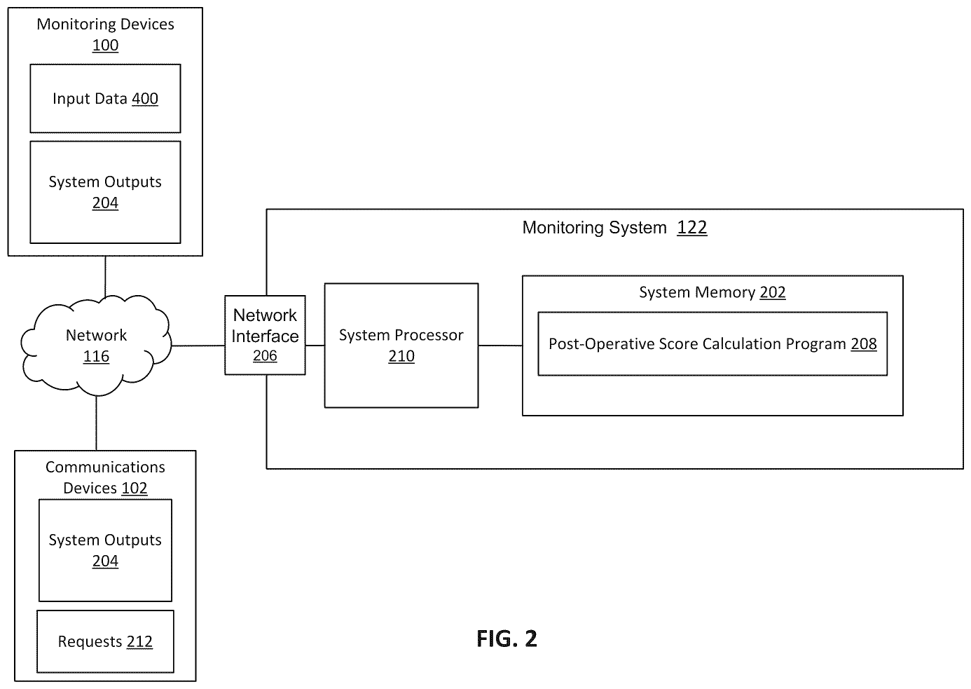

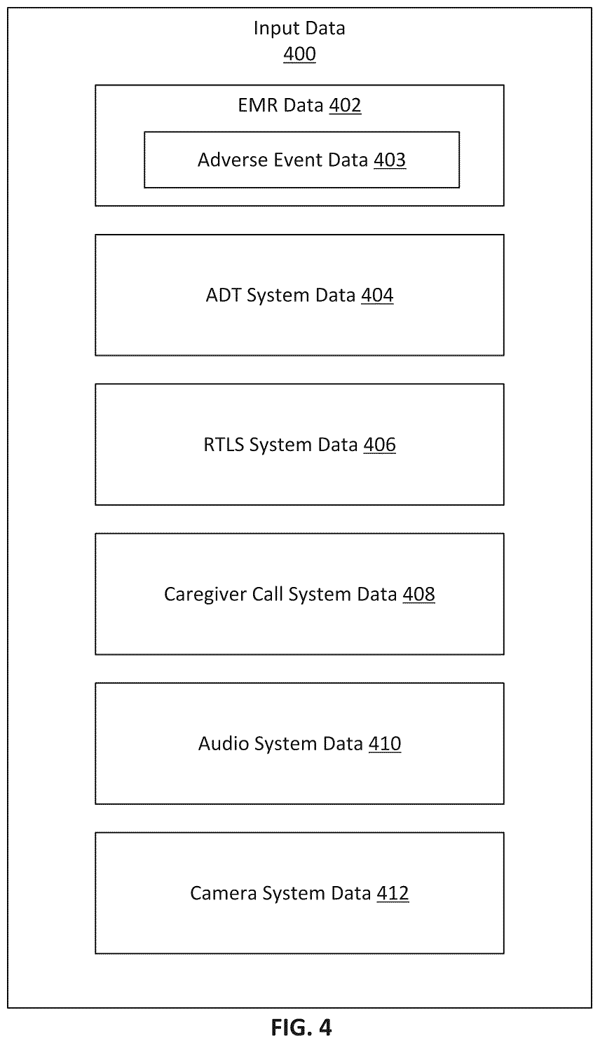

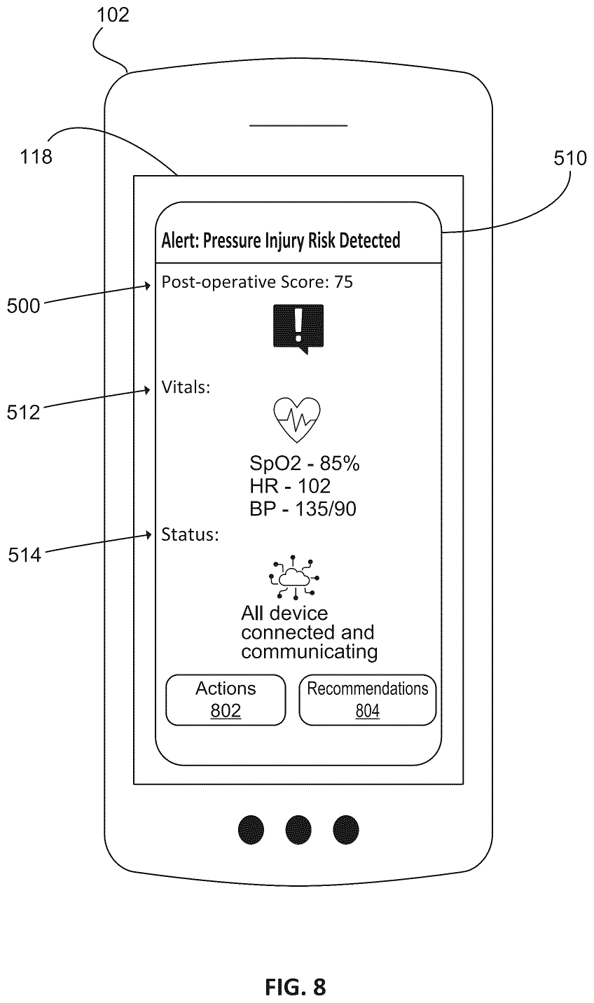

In certain examples, the monitoring system 122 receives input data 400 (as shown in FIG. 4) over the communications network 116 that is generated by the one or more clinical care environment systems 100. The monitoring system 122 analyzes the input data 400 and generates system outputs 204 (as shown in FIGS. 2, 5, 8, and 9) that are transmitted to one or more communications devices 102 via the communications network 116. The system outputs 204 may include one or more post-operative scores 500 (as shown in FIG. 5), alerts 510 (as shown in FIGS. 5 and 8), and recommendations 520 (as shown in FIGS. 5 and 9).

In certain examples, and as described in further detail with respect to FIGS. 5, and 8, the alert is a notification provided to a caregiver C from the monitoring system 122 via the one or more communications devices 102. The alert provides additional information pertaining to one or more system outputs 204 (as shown in FIG. 2). In a non-limiting example, the alert may include a notification sent to a caregiver C to inform the caregiver C that one or more post-operative scores are above a threshold value.

In certain examples, as described below in view of FIGS. 5-9, the recommendation includes one or more suggestions generated by the monitoring system 122 to improve healthcare. In a non-limiting example, the recommendations may include one or more suggestions that are sent to a caregiver C to help the caregiver C improve the one or more post-operative scores when the one or more composite healthcare scores are above the threshold value.

In certain examples, the alert may be sent to the caregiver C with the recommendation to provide information to the caregiver C with one or more suggestions to improve healthcare. In other examples, the alert may be provided to the caregiver C without a recommendation if a recommendation is not needed (e.g., when a caregiver C is already completing the task(s) that would be recommended by the monitoring system 122). In other examples, a recommendation may be provided without an alert to provide a suggestion to improve one or more post-operative scores that are above the threshold value.

In certain examples, the caregiver C may manually request a composite healthcare score that can be provided on demand from the monitoring system 122. The one or more communications devices 102 transmits the request to the monitoring system 122 via the communications network 116. The monitoring system 122 can process the request, generate the alert and/or a recommendation (or update a previous alert and/or recommendation), and transmit the alert and/or recommendation to the communications device 102 via the communications network 116. Furthermore, the caregiver C may provide input to the monitoring system 122 to report a change in the post-operative score after following a recommendation.

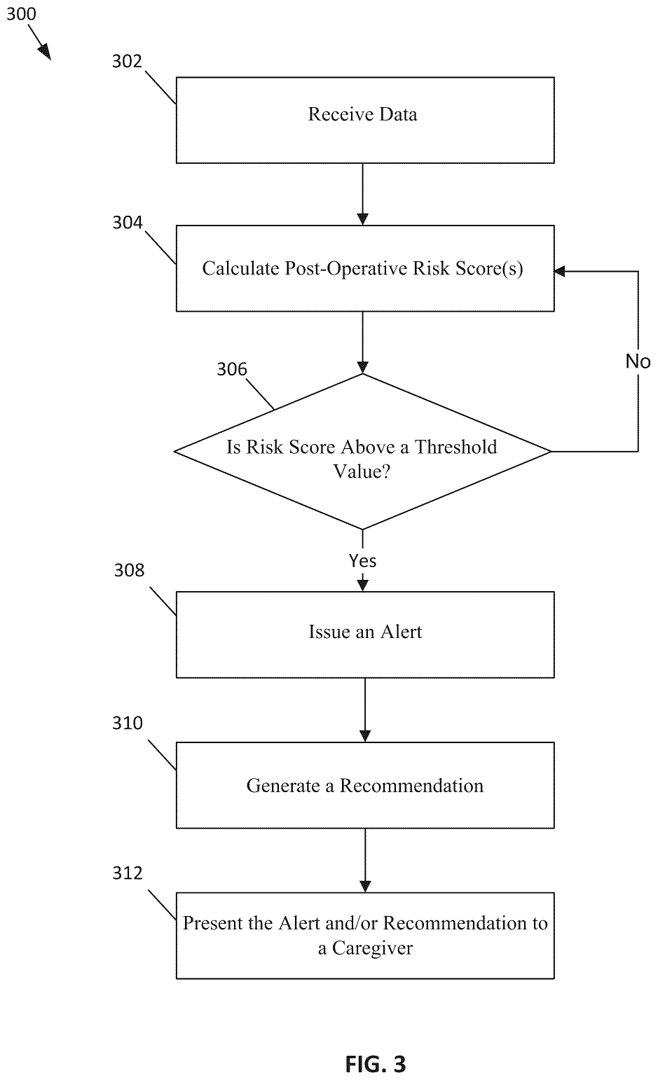

In another example, the monitoring system 122 can proactively generate one or more system outputs 204 and transmit the system outputs 204 to a caregiver C. In certain examples, the monitoring system 122 automatically generates one or more post-operative scores, generates an alert and/or recommendation relating to one or more events that increased the one or more composite healthcare scores above a threshold value, and presents the alert and/or recommendation to a caregiver C (as illustrated and described in further detail with respect to FIGS. 3, 5, 8, and 9). In certain examples, the monitoring system 122 may automatically update one or more system outputs 204 in real time by continuously monitoring input data that is collected by the clinical care environment systems 100. In certain examples, the monitoring system 122 may automatically provide an update to the post-operative score(s) when one or more actions are completed by one or more caregivers C to follow recommendations that are designed to reduce the post-operative score below the threshold value.

Advantages of the monitoring system 122 include improving the prediction of patient(s) P are more susceptible to infection and pressure injuries after surgery, which allows caregivers C properly allocate resources within a clinical care environment 10 based on patient risk. Furthermore, predicting the prediction of which patients P are more susceptible to infection allows caregivers C to implement early intervention strategies that reduce the likelihood of infections and associated complications, create customized care plans that address specific patient risk factors, reduce costs associated with treating infections, and improve the overall health outcomes for patients. Further yet, this greatly decreases the cost associated with infections that are contracted within a clinical care environment 10. Still further yet, this allows the clinical care environments 10 to comply with infection control and monitoring standards that are essential to maintain accreditation and meet regulatory requirements.

As further shown in the example provided in FIG. 1, the caregivers C each wear or otherwise carry a tag 104 that is detectable by antennas 114 (also referred to as RTLS readers) positioned throughout the clinical care environment 10. The antennas 114 are fixed reference points that receive wireless signals from the tags 104. The antennas 114 communicate the wireless signals from the tags 104 to the RTLS 110 via the communications network 116. The RTLS 110 uses the data acquired from the antennas 114 to monitor and track the location of the tags 104 (and of the caregivers C) inside the clinical care environment 10. Tracking the location of a caregiver C is useful for determining which actions a caregiver C performs and the time spent to perform each action. The locations of the caregivers C are stored as RTLS system data 406 (as shown in FIG. 4) within the RTLS 110. The RTLS system data 406 may be transferred to the monitoring system 122 over the communications network 116.

Alternatively, the location of the caregivers C can be monitored by tracking the movement of the communications devices 102. In examples where the communications devices 102 are portable computing devices such as smartphones or tablet computers carried by the caregiver C, the location of the communications devices 102 can be tracked by various tracking techniques including multilateration of signals between cell towers of a telecommunications network and the communications devices 102, or by using geo-spatial positioning techniques by satellite navigation systems such as the Global Positioning System (GPS).

The EMR system 124, also known as electronic health records (EHR) system, is connected to the communications network 116. The EMR system 124 stores the medical history of the patient P. In certain examples, the EMR system 124 generates EMR system data 402 (as shown in FIG. 4) that includes information about a patient's P diagnoses (including a current primary diagnosis), past or current medicines that the patient P is taking, physiological variable measurements of the patient P, clinical interventions provided to the patient P, allergies, immunizations, and treatment plans. EMR system data 402 pertaining to the patient P is useful for calculating composite healthcare scores because it is indicative of the patient P's experience within the clinical care environment 10, including the pain a patient P experiences, the medical interventions the patient P receives, and the outcome of the medical interventions. The EMR system data is illustrated and described in further detail with respect to FIG. 4.

As further shown in the example provided in FIG. 1, a caregiver call system 120 is connected to the communications network 116. The caregiver call system 120 receives requests from the patient P and can generate and send alerts to a caregiver C to respond to the requests from the patient P. In certain examples, the alert can be routed via the communications network 116 to a communications device 102 used by the caregiver C during their shift. In certain examples, the alert can include a request from the patient P to perform a clinical intervention, such as in response to a triggered alarm. In certain examples, the alert can include information retrieved from the EMR system 124 or from an alarm communication system. In certain examples, the caregiver call system 120 includes one or more caregiver call lights (also known as corridor lights or dome lights) outside of a patient room that indicate when a patient P is requesting help from a caregiver C. The caregiver call system data 408 (shown in FIG. 4) is useful because it provides information relating to a patient P's requests, and period of time that elapses before the request is addressed, and a quantity of requests from the patient P.

In the example of FIG. 1, an audio system 126 is shown connected to the communications network 116. The audio system 126 can include a plurality of speakers and microphones positioned throughout the clinical care environment 10 allowing auditory communication between caregivers C, patients P, and the monitoring system 122. The speakers may be included within patient rooms (e.g., on a patient bed 101, along a bed rail of the patient bed 101, or a patient monitoring device such as a spot monitor 103, which is shown in FIGS. 6-7) or within common spaces within the clinical care environment 10 (e.g., hallways, communal spaces, dining areas, etc.). Furthermore, the audio system 126 auditorily provides information pertaining to system outputs 204 to a caregiver C. In certain examples, the audio system 126 records audio from the caregiver C or patient P that indicates the patient P requires assistance.

This may include sounds coming from the patient P that indicate the patient is in distress, verbal communication from the patient P to a caregiver C, or sounds emitted from monitoring equipment, such as the spot monitor 103, that indicate the patient P requires assistance.

In certain examples, the clinical care environment systems 100 include one or more monitoring devices that are internet of things (IoT) medical devices. IoT medical devices may be equipped with sensors, connectivity capabilities to a network, and embedded software to collect, transmit, and analyze data related to patient health and medical conditions. Data collected by IoT medical devices can be sent to caregivers C, other clinical care environment systems, or to the monitoring system 122. In certain examples, the patient bed 101 and spot monitor 103 are IoT medical devices that are connected to the communications network 116.

As further shown in the example provided in FIG. 1, a camera system 128 is connected to the communications network 116. The camera system includes a plurality of cameras (e.g., video cameras, surveillance cameras, etc.) positioned throughout the clinical care environment 10 that monitor movements of individuals. The camera system 128 may include cameras positioned within patient rooms or in common spaces. The camera system 128 may be used to record video that is transmitted to the monitoring system 122 to be used to identify actions performed by the caregiver C or movements from patient P. In certain examples, and as discussed in greater detail below with respect to FIGS. 5-7, the camera system 128 may monitor movements of patients P and caregivers C (including interactions that are made between a patient P and any other individual after surgery).

FIG. 2 schematically illustrates an example of the monitoring system 122 of FIG. 1. The monitoring system 122 is connected to the communications network 116 via a network interface 206 to facilitate a transfer of data to and from the monitoring system 122. In certain examples, the network interface 206 is a physical network interface that is a hardware component installed within a computer or other device. In certain examples, the network interface 206 is a virtual network interface used in virtualized environments that allows a virtualized system to communicate with a physical network.

The monitoring system 122 receives the input data 400 from one or more of the clinical care environment systems 100 over the communications network 116. Examples the clinical care environment systems 100 are illustrated and described in further detail above with respect to FIG. 1. The clinical care environment systems 100 can store the input data 400 and system outputs 204 produced by the monitoring system 122 within a memory.

In certain examples, the monitoring system 122 continuously receives the input data 400 from one or more of the clinical care environment systems 100 and continuously updates the system outputs 204. In certain examples, the monitoring system 122 provides an explanation for why the system outputs 204 were updated. For example, the monitoring system 122 can provide a description of one or more events that were considered in changing the system outputs 204. Examples of events that are considered in changing the system outputs 204 are illustrated and described in further detail with respect to FIGS. 5-7.

A system processor 210 receives and executes instructions from a system memory 202 to perform the functions and aspects described herein. In one example, the system processor 210 receives and executes instructions from a post-operative score calculation program 208. In some examples, the instructions received from the post-operative score calculation program 208 include processing the input data 400 received over the communications network 116. In some examples, processing the input data 400 includes utilizing one or more artificial intelligence models to generate system outputs 204. For instance, a machine learning algorithm can be trained using input data 400 collected from a large number of patients P. Examples of applications using the post-operative score calculation program 208 are illustrated and described in further detail with respect to FIGS. 8-9.

The system memory 202 includes one or more memories configured to store the post-operative score calculation program 208. In certain examples, the system memory 202 stores the input data 400 received via the communications network 116. In such examples, the system memory 202 stores at least one set of input data 400 that includes any combination of the data illustrated and described in further detail with respect to FIG. 4.

In certain examples, the system memory 202 stores the system outputs 204. In such examples, the system memory 202 stores at least some of the system outputs 204 illustrated and described in further detail with respect to FIG. 5.

The system memory 202 can be of various types, including volatile and nonvolatile, removable, and non-removable, and/or persistent media. In some examples, the system memory 202 is an erasable programmable read only memory (EPROM) or flash memory.

The communications device 102 is configured to transmit one or more requests 212 to the monitoring system 122 and receive system outputs 204 via the communications network 116. The one or more requests 212 may be manually generated by a caregiver C when the caregiver C manually requests an alert and/or recommendation from the monitoring system 122 via the one or more communications devices 102. The communications device 102 stores and displays the system outputs 204 for a caregiver C to review. Examples of communications devices 102 are illustrated and described in further detail with respect to FIGS. 8-9.

FIG. 3 schematically illustrates an example of a method 300 of improving post-operative scores. The method 300 can be performed by the monitoring system 122. The method 300 includes a step 302 of receiving data. In some examples, step 302 includes receiving the input data 400 (shown in FIGS. 2 and 4) that is generated by the one or more clinical care environment systems 100. The input data 400 is received by the monitoring system 122 from the one or more clinical care environment systems 100 via the communications network 116 as described and illustrated in further detail with above with respect to FIG. 2. The input data 400 is illustrated and described in further detail below with respect to FIG. 4.

The method 300 includes a step 304 of calculating at least one post-operative score. The at least one post-operative score is calculated by analyzing the input data 400 generated by the one or more clinical care environment systems 100. The input data 400 is analyzed to produce one or more system outputs 204. The system outputs 204 are illustrated and described in further detail with respect to FIG. 5. In certain examples, the at least one post-operative score is calculated based on one or more sub scores including a pressure injury risk score and an infection risk score, which are illustrated and described in further detail with respect to FIG. 5.

The method 300 includes a step 306 of determining whether the post-operative score is above a threshold value. If the post-operative score is above a threshold value, then the monitoring system 122 will proceed by generating a system output 204 (e.g., issuing an alert, generating a recommendation, and presenting the alert and/or recommendation to a caregiver C).

If the post-operative score is below the threshold value, then the monitoring system 122 will proceed by recalculating the post-operative score by analyzing data that is received from the clinical care environment systems 100. In certain examples, the post-operative score is continuously updated in real time by analyzing input data received from the clinical care environment systems 100.

The method 300 includes a step 308 of issuing an alert when the post-operative score is above the threshold value. In certain examples, the alert is transmitted to one or more caregivers C over the communications network 116 to the communications device 102 held by the caregivers C. In certain examples, the alert includes the post-operative score, patient information, and other information. The alert is illustrated and described in further detail with respect to FIGS. 5 and 8.

The method 300 includes a step 310 of generating a recommendation for improving the post-operative score. As discussed below with reference to FIGS. 5-9, the recommendation may include any suggestion for improving the post-operative score. In certain examples, and as illustrated in FIG. 5, the recommendation includes turning the patient bed to reduce the pressure injury risk score, assigning a patient care status to the patient based on a level of care required by the patient P, assigning the patient to a new location or assigning a new caregiver C to the patient to ensure adequate care is provided based on the care status assigned to the patient P, requesting additional patient monitoring from a caregiver C or a monitoring device, or providing other recommendations. In certain examples, one or more recommendations may be implemented automatically by requesting an input from the caregiver via a control (e.g., the communications device 102) to approve the recommendation, receive the input from the caregiver via the control, and automatically implementing the at least one recommendation when the input approves the at least one recommendation. In certain examples, the recommendation is included within the alert that is sent to the caregiver C.

The method includes a step 312 of presenting the alert and/or recommendation to the caregiver C. In certain examples, and as described above with reference to FIGS. 1-2, the alert and/or recommendation may be presented to the caregiver C via a communications device 102 or other devices located within the clinical care environment 10.

FIG. 4 schematically illustrates examples of input data that can be generated by the clinical care environment systems 100 of FIG. 1. As described above, the clinical care environment systems 100 can include the ADT system 108, the RTLS 110, the caregiver call system 120, the EMR system 124, the audio system 126, and the camera system 128. The input data 400 includes EMR system data 402 collected by the EMR system 124, ADT system data 404 collected by the ADT system 108, RTLS system data 406 collected by the RTLS 110, caregiver call system data 408 collected by the caregiver call system 120, audio system data 410 collected by the audio system 126, and camera system data 412 collected by the audio system 126. The monitoring system 122 analyzes one or more of the EMR system data 402, the ADT system data 404, the RTLS system data 406, the caregiver call system data 408, the audio system data 410, and the camera system data 412, including any combinations thereof, to generate system outputs 204, which are described and illustrated with respect to FIGS. 5-9.

The EMR system data 402 includes information about a patient P's medical history, including diagnoses, past or current medicines that the patient P is taking, clinical interventions provided to the patient P, recorded vital sign measurements, and other physiological variable measurements, allergies, immunizations, and treatment plans. In certain examples, the caregiver C can enter the EMR system data 402 into the EMR system 124 using a communications device 102. In certain examples, the EMR system data 402 is utilized to generate system outputs 204 by considering a patient's condition and medical interventions that are provided to the patient.

Furthermore, the EMR system data 402 can include adverse event data 403. The adverse event data 403 includes information relating to adverse events that occur within the clinical care environment 10. Adverse events include, for example, medication errors, surgical complications, HAIs, PIs, falls, diagnostic errors, adverse drug reactions, and other complications that occur to patients P during treatment. The information relating to the adverse events may include, for example, the location(s) where the adverse event occurred and the caregiver(s) assigned to the patient that experienced the adverse event. In certain examples, the monitoring system 122 may calculate the post-operative score by analyzing the adverse event data 403 containing records of one or more locations where an adverse patient event occurred and one or more caregivers C assigned to the patient P when the adverse patient event occurred.

The ADT system data 404 includes data that tracks patients from their moment of arrival at the clinical care environment 10 until their departure, and can also include relevant patient information such as medical record numbers, names, and contact information. In certain examples, the ADT system data 404 can be utilized to generate system outputs 204 by considering the number of patients within the clinical care environment 10, the time each patient P spent within a clinical care environment 10, the location(s) where each patient P stayed within the clinical care environment 10, and any patient transfers that were made between one or more clinical care environments.

The RTLS system data 406 is acquired from the antennas 114 to monitor and track the location of the tags 104 (and the caregivers C) inside the clinical care environment 10. In certain examples, the monitoring system 122 utilizes the RTLS system data 406 to generate the system outputs 204 by monitoring the locations of caregivers C, or any other individuals that are equipped with a tag 104, in real time. In certain examples, the locations of individuals that are tracked with the RTLS 110 is monitored in real-time to update the system outputs 204 and provide one or more updated outputs to a caregiver C or patient P. In certain examples, this includes monitoring the location(s) of one or more caregivers C to determine a period of time spent with one or more patients P.

The caregiver call system data 408 includes requests from a patient P and alerts sent to a caregiver C to respond to the request received from the patient P. In certain examples, the monitoring system 122 can analyze the caregiver call system data 408 to analyze communications between a patient P and caregiver C and calculate a period of time between when a patient P requests care and when the caregiver C provides the care that was requested.

The audio system data 410 can be utilized by the monitoring system 122 to analyze sounds emitted by the patient that indicate signs of distress or communications with a caregiver. Furthermore, the audio system data 410 can be utilized by the monitoring system 122 to identify an auditory request from a caregiver C to provide a system output 204.

The camera system data 412 can be utilized by the monitoring system 122 to analyze movements of a caregiver C or a patient P. Furthermore, the camera system data 412 can be used to analyze a quantity of caregivers C or patients P that enter or exit an operating room 600 or recovery room 700 (as illustrated and described in further detail with respect to FIGS. 6-7). Further yet, the camera system data 412 can be used to analyze a healing progression of a surgical site of the patient P after surgery if the surgical site is viewable by the camera system 128 (i.e., if the surgical site is not covered by bandaging or other materials).

FIG. 5 schematically illustrates examples of system outputs 204 that can be generated by the monitoring system 122 of FIG. 1. The system outputs 204 include one or more of post-operative scores 500, alerts 510, or recommendations 520.

The post-operative scores 500 include at least two scores relating to a pressure injury risk score 502 and an infection risk score 504. The pressure injury risk score 502 and/or the infection risk score 504 are calculated by analyzing the input data 400 received from the clinical care environment systems 100. In certain examples, the post-operative score 500 is a single, composite score calculated by considering one or more of the pressure injury risk score 502 and the infection risk score 504. In certain examples, one or more of the post-operative scores 500, the pressure injury risk score 502, and the infection risk score 504 are a numerical value. The numerical value may include a range of values (e.g., a range from 1-10, 1-100, or any other numerical range).

The pressure injury risk score 502 is a summary of a plurality of risks that indicate how likely the patient P is to develop a pressure injury after surgery. In certain examples, the patient P may be monitored in the patient environment 12 (which includes the operating room 600 (as illustrated in FIG. 6) or a recovery room 700 (as illustrated in FIG. 7) by one or more monitoring devices within the clinical care environment systems 100. As described above, the clinical care environment systems 100 collect input data 400 that may be analyzed by the monitoring system 122. For example, this may include analyzing data collected from the use of one or more load cells 107 (as shown in FIGS. 6-7) positioned on or in a patient bed 101 that the patient P lies on during or after surgery. The load cells may determine any area the patient places significant pressure on that would make the patient P at risk for developing a pressure injury. In another example, the pressure injury risk score may be calculated by viewing one or more body postures of the patient P using the camera system 128 to determine any areas the patient is placing excess pressure on. Furthermore, movements of the patient during surgery, the location of the surgical site, and changes to the wound after surgery may be considered by analyzing the input data 400 recorded from clinical care environment systems 100. For example, the patient information can be recorded by a caregiver C within the EMR System 124, video of the surgical site can be recorded by the camera system 128, and audio can be recorded by the audio system 126 relating to communications involving a patient and one or more caregivers C. Further yet, the ADT system data 404 can be analyzed by the monitoring system 122 to consider patient demographical information that can affect the patient's likelihood of developing a pressure injury or other post-operative complication.

The infection risk score 504 is a summary of a plurality of risks that indicate how likely the patient P is to develop an infection after surgery. In certain examples, data used to calculate the pressure injury risk score 502 may also be used to calculate the infection risk score. For example, factors such as a period of time a patient spends on an operating table, movements of the patient during surgery, the location of a surgical site, pain the patient P is experiencing, the presence of erythema, odor, or exudate, and patient demographical information may be considered when calculating the pressure injury risk score 502 or the infection risk score 504.

Furthermore, the infection risk score 504 may be at least partially calculated by monitoring interactions between a patient P and other persons (e.g., other patients P or caregivers C). Monitoring interactions as described herein is broadly used to connote, without limitation, the automated collection of data characterizing any interaction with a patient, whether by in-room monitoring (e.g., audio and/or video and/or patient monitoring systems and/or other proximity sensing techniques) or personal device tracking (e.g., mobile phone or other device) or system monitoring (e.g., surveillance of other automated data systems such as EMR, nurse call systems, and the like which describe such interactions). In certain examples, and as described in further detail with respect to FIGS. 6-7, input data 400 can be analyzed by the monitoring system 122 to determine a quantity of persons that entered or exited a surgical environment. It is desirable to determine the quantity of persons that entered or exited the surgical environment because larger quantities of persons may negatively impact the sterility of the surgical environment, especially if proper precautions are not made to maintain the integrity of a sterile surgical environment. The input data 400 can also be analyzed by the monitoring system 122 to determine a quantity of persons in the surgical environment at any time while before, after, or during a surgery. Further yet, the input data can be used to analyze historical risks with a specific surgical environment or with specific caregivers (i.e., the monitoring system 122 can determine which surgical environment(s) or caregiver(s) are most commonly associated with pressure injuries or infections that occur within the clinical care environment 10.

In certain examples, the interactions are automatically monitored using one or more devices within the patient environment 12. The one or more devices can include at least one of the audio system 126, the camera system 128, and the RTLS 110. Furthermore, in certain examples, the interactions are automatically monitored using one or more communications devices 102. As described above with reference to FIG. 1, the one or more communications devices 102 can include a smartphone, a tablet computer, and other portable computing devices.

The alerts 510 include one or more post-operative scores 500 (as discussed above), patient information 512, and other information 514. In certain examples, the monitoring system 122 transmits one or more alerts 510 to communications device(s) 102 that are accessible to one or more caregivers C. An example of an alert 510 that may be transmitted to the communications device(s) 102 that are accessible to one or more caregivers C is illustrated and described in further detail with respect to FIG. 8.

The patient information 512 includes information that is relevant to the patient's treatment within the clinical care environment 10. This may include EMR system data 402 or ADT system data 404. Furthermore, the alerts 510 may include other information 514 such as an operational status of one or more monitoring devices within the clinical care environment.

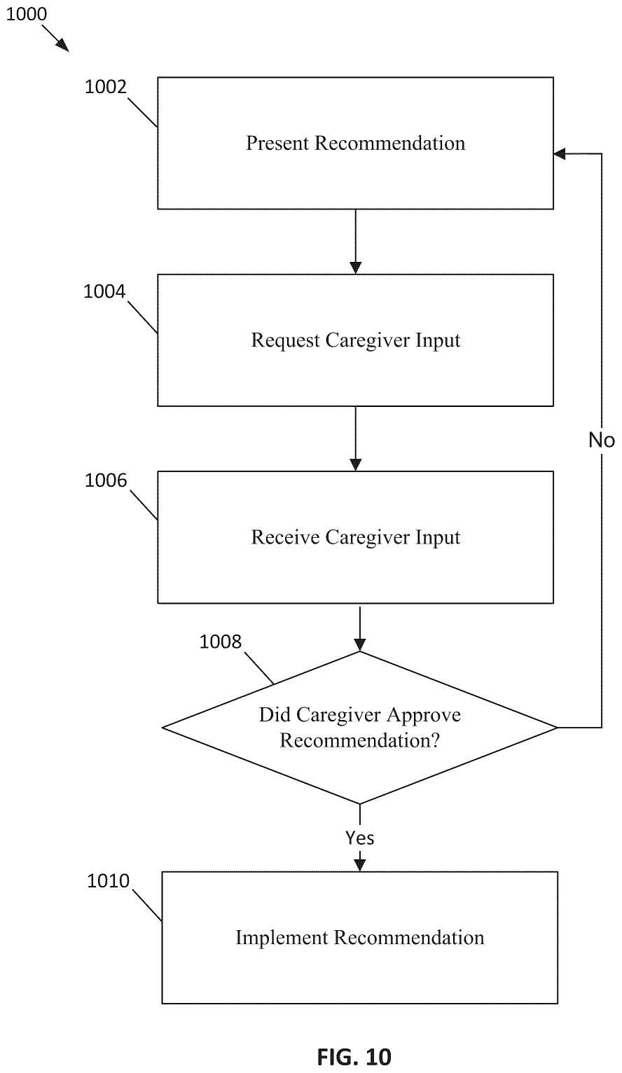

The recommendations 520 can include at least one suggestion to improve a post-operative score relating to the patient P. In certain examples, the recommendation 520 addresses concerns that are communicated within the alert 510. The monitoring system 122 transmits one or more recommendations 520 to communications device(s) 102 that are accessible to one or more caregivers C. The recommendations 520 may include, but are not limited to, the following examples. Furthermore, in certain examples, the recommendations may be automatically implemented by the monitoring system 122. A process for automatically implementing one or more recommendations 520 is described and illustrated in further detail with respect to FIG. 10.

In a first example, the recommendations 520 may include turning the patient bed 522 to reduce the pressure injury risk score 502. The recommendation to turn the patient bed 522 may be provided to a caregiver C when the pressure injury risk score 502 is above a threshold value. Turning the patient bed 101 is desirable when the patient P is at risk of a pressure injury because it helps distribute pressure and reduce the risk of skin breakdown, which, in turn, can lead to painful and potentially serious infections. The monitoring system 122 may automatically turn a patient bed 101 when the post-operative score 500 is above a threshold value by communicating with the one or more medical devices 14, including the patient bed 101.

In a second example, the recommendations 520 may include assigning a patient care status 524 to the patient P. The patient care status may be assigned to a patient P based on a level of care that is required by the patient P. The level of care required by the patient P may be determined by the monitoring system 122 by analyzing the input data 400. The input data 400 can be analyzed by extracting patient information stored within the EMR system data 402 and the ADT system data 404. The care required by a patient P may depend upon the frequency at which caregivers C provide care to the patient P, which can be determined by tracking the locations of caregivers C over time by analyzing the RTLS system data 406, audio system data 410, or camera system data 412 or by monitoring patient interactions with the caregivers C by analyzing the caregiver call system data 408, the audio system data 410, or the camera system data 412. The patient P may be assigned a patient care status corresponding to a higher level of required care if the post-operative score 500 exceeds a threshold value. Conversely, the patient P may be assigned a patient care status corresponding to a lower level of required care if the post-operative score 500 is below the threshold value.

In a third example, the recommendations 520 include requesting increased patient monitoring 526. Increased patient monitoring can be requested if the patient requires additional care than what is currently being provided. For example, the patient P may require increased monitoring from a caregiver C when the post-operative score 500 is above the threshold value. Conversely, the patient P may require decreased monitoring from the caregiver C when the post-operative score is below the threshold value.

In a fourth example, the recommendations 520 include assigning a patient P to a caregiver 528. The patient P can be assigned to the caregiver C based on the patient care status of the patient P. For example, patients with a patient care status corresponding to a higher degree of care required can be assigned to caregivers C with greater availability to care for patients (i.e., caregivers C that have a reduced workload relative to other caregivers C within the clinical care environment 10). Alternatively, patients with a lower degree of care can be assigned to caregivers with less availability. In certain examples, the patients P may be exchanged between caregivers C to evenly distribute caregiver workloads among the caregivers C.

Furthermore, the recommendations 520 may include any number of other recommendations 530 to reduce the post-operative score(s) of one or more patients P. For example, the other recommendations 530 may include a recommendation to move the patient P to a different patient environment 12 or assign a new caregiver C to the patient if the monitoring system 122 analyzes the adverse event data 403 and associates the patient environment 12 or the caregiver C with number of adverse events that are above a threshold value.

FIG. 6 illustrates an example of an operating room 600 equipped with one or more of the clinical care environment systems 100 to collect input data 400 that can be analyzed by the monitoring system 122 to generate system outputs 204. The operating room 600 is located within the clinical care environment 10. The operating room includes one or more clinical care environment systems 100, such as the camera system 128, the RTLS 110 (including one or more antennas 114 and tags 104 as shown in FIG. 6), and the audio system 126. The one or more clinical care environment systems 100 are configured to collect input data 400 relating to surgeries or other treatments that are provided to the patient P within the operating room 600.

In the example shown, the patient P is lying on a surgical table 604 in a supine position. The patient P may be covered by a patient covering 606, such as a sterile surgical drape, sterile surgical sheets, or other sterile covers. Furthermore, the surgical table 604 may be equipped with one or more load cells 107 for measuring a distribution of the patient's weight during surgery, which can be analyzed by the monitoring system to determine areas of the patient P that are at risk of developing a pressure injury.

The operating room includes one or more medical devices 14, such as the patient bed 101, the spot monitor 103, the infusion pump 109, and the contact-free continuous monitoring system 132. The spot monitor 103 measures and displays vital signs of the patient P, such as the patient's blood pressure, pulse rate, oxygen saturation, temperature, respiratory rate, and other measurements. The infusion pump 109 delivers fluids, medications, nutrients, and other therapeutic substances to the patient's body in a controlled manner. The contact-free continuous monitoring system 132 monitors the patient P for signs of deterioration and provides visual indications of patient deterioration to a caregiver. In certain examples, the signs of visual deterioration include visual projections along a floor next to the patient bed 101. The contact-free continuous monitoring system may be an extension of the Continuous Patient Monitoring Device with EarlySense® platform available from Hillrom®.

As discussed above with reference to FIG. 5, the clinical care environment systems 100 may be used to analyze interactions between the patient P and other persons. In certain examples, the interactions are monitored by at least one of measuring a time the patient spends on the surgical table 604 during a surgery, detecting a quantity of caregivers C that entered a surgical environment (i.e., the operating room 600) where the surgery was performed on the patient P, and monitoring one or more patient movements during or after the surgery.

Furthermore, the post-operative scores 500 may be calculated by analyzing the one or more patient movements and one or more caregiver movements during the surgery. In certain examples, analyzing the one or more patient movements and the one or more caregiver movements during the surgery includes at least one of measuring a time the patient spends on the surgical table during the surgery and a quantity of caregivers that entered the surgical environment while the surgery was performed on the patient. For example, the number of caregivers C1, C2 that enter or exit the operating room 600 through an entrance/exit 602 may be tracked by using the monitoring system 122 to analyze the RTLS system data 406, audio system data 410, and/or the camera system data 412.

FIG. 7 illustrates an example of a recovery room 700 equipped with one or more of the clinical care environment systems 100 to collect input data 400 that can be analyzed by the monitoring system 122 to generate system outputs 204. The recovery room 700 (a.k.a. a post-anesthesia care unit or a post-operative recovery room) can be equipped with many of the devices described above in FIG. 6. After surgery, the patient P is transferred to the recovery room and the patient's vital signs are monitored. For example, the patient's vital signs can be monitored using the spot monitor 103, and medications, fluids, and other nutrients may be supplied to the patient via the infusion pump 109. The patient P may be monitored by one or more caregivers C as the patient recovers from the surgery. In certain examples, one or more caregivers C and/or the camera system 128 may be used to monitor the surgical site of the patient and the surgical site progression as the surgical site heals after surgery. For example, if the surgical site is not covered by a patient covering, the camera system 128 may record images of the surgical site as the patient recovers from the surgery to document the surgical site healing progression. Furthermore, the one or more caregivers C may record information within the EMR system 124 regarding the surgical site healing progression as the patient P recovers from surgery.

Additionally, the incontinence pad 134 may be positioned under the patient to absorb moisture from an incontinence event. The patient bed 101 may include one or more sensors for detecting moisture from an incontinence event. Furthermore, the camera system 128 and monitoring system 122 may include elements of the systems and methods described in U.S. Provisional Application 63/497,668, filed Apr. 21, 2023, titled “Detection of Incontinence Events, which are incorporated by reference in their entireties, such as an infrared lens for recording a heat signature from an incontinence event, and a system for identifying an incontinence event, alerting caregivers, and providing recommendations to care for the patient P.

FIG. 8 illustrates an example of a communications device 102 displaying an alert 510 that the post-operative score 500 is above a threshold value. In this example, the alert 510 is displayed on a display 118 of the communications device 102. The display 118 can include a touchscreen that displays outputs and receives tactile inputs from the caregiver C.

As illustrated and described in further detail with respect to FIG. 5, the alert 510 further includes patient information 512 such as patient vital signs. In this example, the patient vital signs include the oxygen saturation of the patient (e.g., 85%), the patient's heart rate (e.g., 102 beats per minute), and the patient's blood pressure (e.g., a systolic blood pressure of 135 and a diastolic blood pressure of ninety). Furthermore, the alert includes other information 514 indicating all of the one or more medical devices 14 are properly connected to the network. In certain examples, the one or more medical devices 14 include Internet of Things (IoT) devices as described above with reference to FIG. 1.

Furthermore, the alert 510 can include an actions icon 802 and a recommendations icon 804 for requesting additional information from the monitoring system 122 or requesting a recommendation 520. In certain examples, the actions icon 930 allows the user (i.e., a caregiver C) to request an update to the system output 204 or implement one or more recommendations 520 to improve the one or more post-operative scores. The recommendations icon 940 allows a caregiver C to view one or more recommendations 520 for improving the one or more post-operative scores. An example of a recommendation 520 that is displayed when the recommendations icon 804 is selected is described and illustrated with respect to FIG. 9.

FIG. 9 illustrates an example of a communications device 102 displaying a recommendation 520 corresponding to the alert 510 in FIG. 8 to improve the post-operative score 500. In this example, the recommendation 520 relates to a recommendation of turning the patient bed 522 as illustrated and described above with reference to FIG. 5. A description 904 is included that recommends an action for the caregiver C to complete to inspect the patient for pressure injuries and turn the patient. This recommendation 520 may be issued when the post-operative score 500 exceeds the threshold value or if a predetermined time elapses with the patient P lying in the same position.

The recommendation 520 may include a request to a caregiver C via a control on a graphical user interface of the communications device 102 to approve the recommendation 520. The caregiver C can provide an input to the monitoring system 122 by selecting a yes icon 908 (which approves the recommendation 520) or a no icon 902 (which rejects the recommendation 520). The monitoring system 122 receives the input from the caregiver and, if the caregiver approves the recommendation 520, the monitoring system 122 may automatically implement the recommendation (e.g., by automatically turning the patient bed 101 to adjust the patient P and reduce the post-operative score 500). A process for automatically implementing one or more recommendations 520 is illustrated and described in further detail with respect to FIG. 10.

Furthermore, the recommendation 520 may include additional patient information 909 that the user can retrieve by selecting one or more icons. In this example, the user may select an EMR System Data icon 910 to view the EMR system data 402 and an ADT system data icon 912 to view the ADT system data 404. This data may be useful to gather basic information about the patient P such as the patient's location within the clinical care environment 10, patient demographical information, and the patient's medical records.

Further yet, the recommendation 520 may include additional actions 914 that may be requested by selecting an additional actions icon 916. In certain examples, the additional actions icon 916 generates a request for the monitoring system 122 to generate additional recommendations and/or update information that was previously provided to the user.

FIG. 10 illustrates an example of automatically implementing at least one of the recommendations 520 to improve a post-operative score 500. The method 1000 includes a step 1002 of presenting a recommendation 520 to a caregiver C. In certain examples, the monitoring system 122 generates the recommendation 520 and presents the recommendation 520 to the caregiver C by transmitting the recommendation 520 to the communications device 102 via the communications network 116.

The method 1000 includes a step 1004 of requesting caregiver input from one or more caregivers C. In certain examples, the request is sent to the caregiver C via a communications device 102 where the caregiver may provide input by selecting one or more icons on a display of the communications device (as illustrated and described in further detail with respect to FIG. 9).

The method includes a step of 1006 of receiving the caregiver input from the caregiver C. In certain examples, the caregiver input is received via a selection of one or more icons on the display of the communications device 102, wherein the communications device transmits the selection to the monitoring system 122 via the communications network 116.

The method includes a step 1008 of determining whether the caregiver C approved the recommendation 520. If the caregiver C approved the recommendation (i.e., “Yes” in step 1008), the monitoring system 122 may proceed to a step 1010 of automatically implementing the recommendation as described and illustrated in further detail with respect to FIG. 9. If the caregiver does not approve the recommendation (i.e., “No” in step 1008), then the monitoring system 122 may present a new recommendation to the caregiver C and repeat one or more of the steps 1002, 1004, 1006, and 1008.

The systems and methods described herein provide significant technical advantages. For example, the monitoring system 122 improves the efficiency of systems for identifying post-operative risks in clinical care facilities, including risks of infection and pressure injuries. Furthermore, the monitoring system 122 is a practical application in healthcare technology that conveys specific alerts and/or recommendations, which may include graphical and/or visual information, in a specific way at a communications device 102 to assist caregivers C in identifying and treating post-operative infections and pressure injuries. Further yet, the monitoring system 122 may automatically implement one or more recommendations to improve the functioning of healthcare systems, such as to improve one or more post-operative scores 500 by turning a patient, assigning a patient care status, requesting increased patient monitoring, assigning a patient to a caregiver, or reassigning a patient to a new caregiver or patient environment based on adverse event data that is associated with a caregiver or patient environment.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless of whether shown and described in combination or separately, the various features (both structural and methodological) are intended to be selectively included or omitted to produce an embodiment with a particular set of features.

Having been provided with the description and illustration of the present application, one skilled in the art may envision variations, modifications, and alternate embodiments falling within the spirit of the broader aspects of the claimed inventions and the general inventive concepts embodied in this application that do not depart from the broader scope.

The various embodiments described above are provided by way of illustration only and should not be construed to be limiting in any way. Various modifications can be made to the embodiments described above without departing from the true spirit and scope of the disclosure.

What is claimed is:

1. A system for improving a prediction of post-operative patient risks, the system comprising:
at least one processing device; and
a memory device storing instructions which, when executed by the at least one processing device, cause the at least one processing device to:
receive data from one or more monitoring devices;
calculate a post-operative score based on the data received from the one or more monitoring devices, wherein the post-operative score is at least partially calculated by monitoring interactions between a patient and other persons;
issue an alert when the post-operative score exceeds a threshold value;
generate at least one recommendation for improving the post-operative score; and
present a control on a graphical user interface for viewing the post-operative score or the at least one recommendation.

2. The system of claim 1, further comprising one or more medical devices connected to a network, and wherein the one or more medical devices include at least one of a patient bed, a spot monitor, a contact-free continuous monitoring device, and an infusion pump.

3. The system of claim 1, wherein the post-operative score includes a pressure injury risk score, and the pressure injury risk score is calculated by analyzing the data, and the at least one recommendation includes turning a patient to reduce the pressure injury risk score.

4. The system of claim 1, wherein the post-operative score includes an infection risk score calculated based on at least a quantity of caregivers that entered a surgical environment.

5. The system of claim 1, wherein the instructions, when executed by the at least one processing device, further cause the at least one processing device to:
automatically implement the at least one recommendation when an input approving the at least one recommendation is received.

6. The system of claim 1, wherein the at least one recommendation includes assigning a patient care status to the patient based on a level of care required by the patient.

7. The system of claim 1, wherein the interactions include at least one of measuring a time the patient spends on a surgical table during a surgery, detecting a quantity of caregivers that entered a surgical environment during the surgery, and monitoring one or more patient movements during or after the surgery.

8. The system of claim 7, wherein the post-operative score is calculated based on the time the patient spends on the surgical table during the surgery, the quantity of caregivers that entered the surgical environment during the surgery, and the one or more patient movements.

9. The system of claim 1, wherein the interactions are automatically monitored using one or more devices within a patient environment, and the one or more devices include at least one of a microphone, a camera, and a real-time location system (RTLS) tag.

10. The system of claim 1, wherein the interactions are automatically monitored using one or more communications devices that include a smartphone or a tablet computer.

11. The system of claim 1, wherein the post-operative score is calculated by analyzing electronic health records pertaining to the patient, wherein the electronic health records include at least one of the patient demographics, a description of a surgery site, a site progression description of the surgery site during and after the surgery, the patient's medical history, the patient's diet, and observations made before, during, or after the surgery.

12. The system of claim 1, wherein the post-operative score is calculated by analyzing adverse event data including a location where an adverse patient event occurred and one or more caregivers assigned to the patient when the adverse patient event occurred.

13. The system of claim 1, wherein the recommendation includes assigning the patient to a new location or assigning a new caregiver to the patient.

14. A method for improving a prediction of post-operative infection and pressure injuries, the method comprising:
receiving data from at least one monitoring device;
calculating a post-operative score based on the data received from the at least one monitoring device, wherein the post-operative score is at least partially calculated by monitoring interactions between a patient and other persons;
issuing an alert when the post-operative score exceeds a threshold value;
generating at least one recommendation for improving the post-operative score; and
presenting a control on a graphical user interface for viewing at least one of the post-operative score and the at least one recommendation.

15. The method of claim 14, further comprising:
automatically implementing the at least one recommendation when an input approving the at least one recommendation is received.

16. The method of claim 14, wherein the at least one recommendation includes turning a patient to reduce a risk of a pressure injury.

17. The method of claim 14, wherein the at least one recommendation includes assigning a patient care status to the patient based on a level of care required by the patient.

18. The method of claim 14, wherein the at least one recommendation includes:
calculating a first workload score for an assigned caregiver;
calculating a second workload score for an unassigned caregiver; and
assigning the patient to the unassigned caregiver when the second workload score is less than the first workload score.

19. The method of claim 14, wherein the post-operative score is calculated based on one or more of a time spent on a surgical table during the surgery, a quantity of caregivers that entered a surgical environment, and patient movement.

20. A non-transitory computer readable storage medium storing instructions, which when executed by a computing device, causes the computing device to:
receive data from at least one monitoring device;
calculate a post-operative score based on the data received from the at least one monitoring device, wherein the post-operative score is at least partially calculated by monitoring interactions between a patient and other persons;
issue an alert when the post-operative score exceeds a threshold value;
generate at least one recommendation for improving the post-operative score; and
present a control on a graphical user interface for viewing at least one of the post-operative score and the at least one recommendation.
